(19) United States
(12) Patent Application Publication
(10) Pub. No.: US 2020/0157506 A1
(43) Pub. Date: May 21, 2020

(54) Methods of Generating Mycelial Scaffolds and Applications Thereof

(71) Applicant: Ecovative Design LLC, Green Island, NY (US)

(72) Inventors: Eben Bayer, Troy, NY (US); Gavin McIntyre, Troy, NY (US); Peter Mueller, Poestenkill, NY (US); Meghan O'Brien, Halfmoon, NY (US); Damen Schaak, Halfmoon, NY (US); Jacob Winiski, Troy, NY (US); Alex Carlton, Troy, NY (US)

(73) Assignee: Ecovative Design LLC, Green Island, NY (US)

(21) Appl. No.: 16/688,699

(22) Filed: Nov. 19, 2019

Related U.S. Application Data
(60) Provisional application No. 62/769,789, filed on Nov. 20, 2018.

(51) Int. Cl. C12N 5/077; C12N 1/14; A23J 3/22

(52) U.S. Cl. CPC C12N 5/0658; C12N 2501/10; A23J 3/227; C12N 1/14

(57) ABSTRACT

Several methods are described for generating mycelial scaffolds for use several technologies. In one embodiment, a mycelial scaffold is generated using a perfusion bioreactor system for cell-based meat technologies. In another embodiment, a mycelial scaffold is prepared for biomedical applications. The mycelial scaffolds may be generated from a liquid medium or from a solid substrate.

This is a Non-Provisional Patent Application and claims the benefit of Provisional Patent Application 62/769,789 filed Nov. 20, 2018.

This invention relates to methods of generating mycelial scaffolds. More particularly, this invention relates to methods of generating biocompatible and biodegradable mycelial scaffolds.

### BACKGROUND

As is known, filamentous fungi are comprised of cross-linked networks of filamentous cells called hyphae, which expand via polarized tip extension and branch formation (increasing the number of growing tips), which is equivalent to cell division in animals and plants. See Griffin D, Timberlake W, Cheney J., Regulation of macromolecular synthesis, colony development and specific growth rate of Achlya bisexualis during balanced growth. Journal of General Microbiology 80, 381-388. (1974). Hyphal tip extension can display a number of tropisms (positive or negative) including gravitropisms, autotropisms, and galvanotropisms, of which modification is adequate to affect meaningful organizational and morphological variety in the fungal thallus (mycelium) and fruiting bodies (mushrooms) See Moore, Fungal Morphogenesis. Cambridge University Press. Cambridge, UK. (1998).

Filamentous fungi are defined by their phenotypic plasticity and may produce a secondary mycelium which, based on the “fuzzy logic” of differentiation as a function of differential expression of discrete “subroutines” rather than defined pathways (See, Moore, Tolerance of Imprecision in Fungal Morphogenesis. Proceedings of the Fourth Conference on the Genetics and Cellular Biology of Basidiomycetes, 13-19), can express variable degrees of differentiation spanning from complex reproductive structures (mushrooms) to a completely undifferentiated vegetative mycelium expressing a variety of network morphologies varying in cell density, branching/crosslinking frequency, cell diameter distribution, cellular agglomeration, structural anisotropy, and volume fraction.

As described in U.S. Ser. No. 16/190,585, filed Nov. 14, 2018, one known method of growing a biopolymer material employs incubation of a growth media comprised of nutritive substrate and a fungus in containers that are placed in a closed incubation chamber with air flows passed over each container while the chamber is maintained with a predetermined environment of humidity, temperature, carbon dioxide and oxygen.

As described in U.S. Ser. No. 16/519,384, a panel of biopolymer material as described in U.S. Ser. No. 16/190,585, may be modified to generate a material with a custom texture, flavor, and nutritional profile for use as a foodstuff or a tissue scaffold. The method involves tailoring the density, morphology, and composition of the undifferentiated fungal material during growth and/or the use of post-processes, to improve mouth-feel and/or affinity toward flavors, fats, cellular cultures, or the like.

In one embodiment, the growth conditions in the incubation chamber are altered to yield a well-aligned macromolecular structure, resembling meat, which can then be amended with flavorings and other additives including, but not limited to, proteins, fats, flavors, aromatics, heme molecules, micronutrients, and colorants.

As is known, cell-based meat technologies generally employ perfusion bioreactor systems consisting of suspension reactor units for beef myocyte propagation, dialysis, oxygenation, pumps for media cycling between reactor units and media feeding, and scaffold bioreactor units for producing agglomerated cell masses with or without mechanical actuation of the agglomerated cellular mass. WO2018011805A9 (Nahmias), JP6111510B1 (Yi) and Byrd, Clean meat's path to your dinner plate, The Good Food Institute. Website Accessed Nov. 14, 2018, https://www.gfi.org/clean-meats-path-to-commercialization.

As is also known, tissue cultivation and engineering for biomedical applications focused on production or repair of damaged organs typically require cultivation of given cells on scaffolds of particular mechanical, porosity, biocompatibility and biodegradability characteristics.

It is an object of the invention to leverage the phenotypic plasticity of filamentous fungi to produce fungal scaffold materials with specifically targeted network morphologies.

It is another object of the invention to produce mycelium scaffolds for implementation in perfusion bioreactor systems for cell-based meat technologies.

It is another object of the invention to provide mycelium scaffolds that provide an optimized fibrous, complex substrate for adhesion, propagation, and agglomeration of mammalian cells in suspended or submerged culture.

It is an object of the described invention to produce biocompatible and biodegradable mycelium scaffolds with unique plasticity of manufacture, allowing for porosity and structure to be uniquely tunable for biomedical applications.

### BRIEF DESCRIPTION OF THE INVENTION

Briefly, the invention provides a method of generating a mycelial scaffold comprising the step of placing a substrate of a nutritive substrate and a fungus in a defined environment with a temperature of from 85° F. to 95° F. and a carbon dioxide content of from 3% to 7% of the environment wherein the fungus is characterized in being a. biocompatible species.

Thereafter, the substrate is incubated in the environment to induce mycological biopolymer growth from the substrate without producing a stipe, cap or spore therein and then the growth of mycological biopolymer is removed from the substrate as a one piece self-contained billet.

The methods described within can be used to modify a three-dimensional mycelial matrix, as described in “Mycological Biopolymers Grown in Void Space Tooling” (US 20150033620 A), to create a custom, mass-produced, non-animal scaffold as a stand-alone material, or as a structural scaffold for cultivation of a non-filamentous secondary cell-type.

The methods allow for the production of large, inert, tissue billets that can be further modified to generate a material with custom texture, flavor, and nutritional profile for use in biomedical applications or as a foodstuff. The methods involve tailoring the density, morphology, and composition of the fungal hyphal matrix during growth and/or the use of post-processes.

One embodiment of this involves altering incubation conditions to yield a well-aligned macromolecular structure, resembling meat, which can then be amended with flavorings and other additives (including, but not limited to, proteins, fats, flavors, aromatics, heme molecules, micronutrients, and colorants).

A second embodiment involves the deposition of flavorings and other additives during the growth process, either through liquid or solid deposition, or through natural cellular uptake (bio-adsorption) (e.g., increasing mineral content in growth media, to increase final content in tissue).

A third embodiment involves the removal of unwanted residues (e.g., malodors, enzymes that affect shelf-stability, etc.) through either post-processing, or the altering of incubation conditions.

A fourth embodiment involves the tuning of incubation, synthetic biology, and/or post-process conditions to yield a tissue that, texturally, resembles animal meat (e.g., increasing alignment and decreasing growth density via temperature and airflow controls and/or mechanically, enzymatically, or chemically altering the structure of the tissue).

A fifth embodiment involves using this latter tissue (whole, or washed of any interfering residues) as a three-dimensional matrix in which non-fungal tissue cells can be supported and cultured, allowing for the in vitro production of tissue for meat consumption, or biomedical applications. This tissue can be engineered, using growth conditions, post-processing, or synthetic biology to increase the affinity for desired cell growth (e.g., increasing or decreasing porosity, increasing or decreasing mycelial diameter, deacetylation of the chitin, enhanced cell adhesion sites, or improving yield by generating more limiting nutrients and the like).

These and other objects and advantages of the invention will become more apparent from the following detailed description, taken with the accompanying drawings wherein:

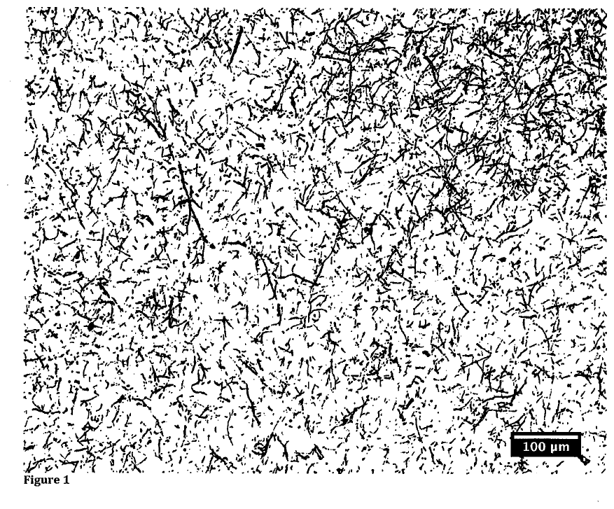

FIG. 1 illustrates a photomicrograph of a vegetative mycelium comprised of an isotropic matrix of discrete hyphae during growth;

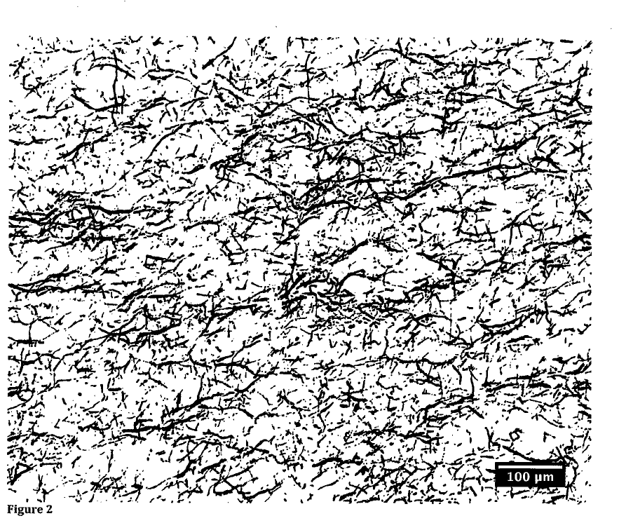

FIG. 2 illustrates a photomicrograph of a modified isotropic matrix with increased strand thickness and increased network fractional anisotropy in accordance with the invention;

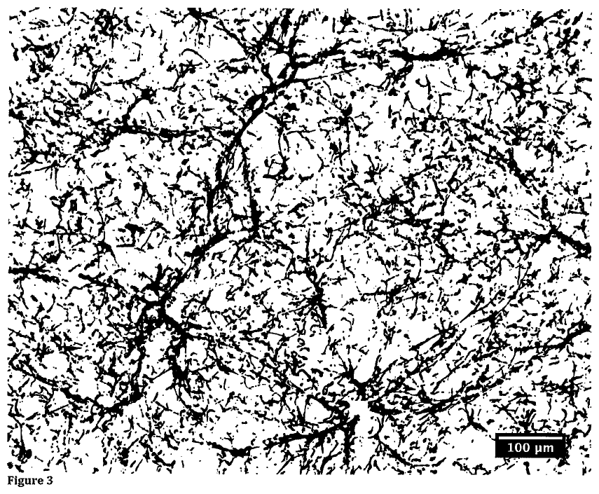

FIG. 3 illustrates a photomicrograph of a modified isotropic matrix with increased strand thickness and without increased network fractional anisotropy in accordance with the invention;

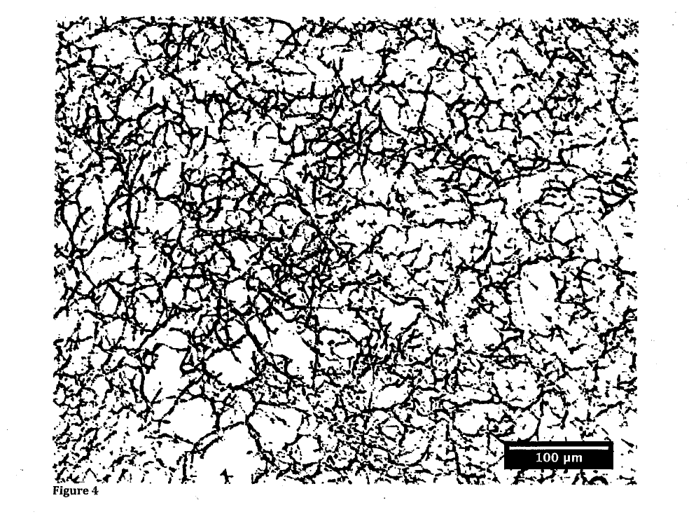

FIG. 4 illustrates a photomicrograph of a modified isotropic matrix with an expressed ellipsoidal morphology in accordance with the invention; and

FIG. 5 illustrates a flow diagram of an apparatus employing a myocyte suspension reactor unit with a filamentous scaffold tray unit for attaching mycocytes to a hyphal scaffold within the tray unit.

### EXAMPLE METHODS

Static Submerged-Submerged Cultivation for Production of Composite Cellular Masses

1. Filamentous organism inoculum is introduced into a bioreactor vessel containing a liquid medium prepared with appropriate asepsis and nutrition for cultivation of the given filamentous species, and may or may not contain a solid substrate or surface to support filamentous growth, creating a first inoculated media. An example liquid medium appropriate for Laetiporus spp. would be 20 g/L malt extract with 2 g/L peptone. The media may be filter sterilized via a 0.2 um filter or pressure sterilized at 15 psi for 45 minutes.

2. The first inoculated media is incubated under conditions selected to affect a specific three-dimensional filamentous network morphology. A generic example for Laetiporus spp. would be static incubation at 27° C. for 15 days. If a solid substrate or surface is included in the vessel, the three-dimensional filamentous network will develop with attachment to the surface, if not the filamentous network will develop within the volume of the vessel. A suitable substrate would have pore sizes >1 um, such that hyphae can penetrate the substrate.

3. After development of the three-dimensional filamentous network has concluded, the culture media within the vessel is replaced with chemistry designed to decellularize the hyphal matrix, retaining the structural wall matrix of the fungal cells while removing all components with the potential to interfere in non-filamentous cell growth, creating a decellularized filamentous scaffold. The chemistry employed is an immersion in a solvent, particularly a 75% ethanol solution for a period greater than 1 hour. The solvent and effluent are then rinsed away with deionized water.

4. After decellularization, the decellularization chemistry is replaced with an appropriate liquid medium for cultivation of a given cell line of non-filamentous organism, and inoculum of the non-filamentous organism introduced into the vessel creating a second inoculated media.

5. The second inoculated media is incubated under conditions appropriate to support metabolism and growth of the given line of non-filamentous organism within the filamentous scaffold (e.g. typical conditions for cultivating myocytes), populating the inter-cellular regions of the filamentous scaffold and attaching to the surface of the decellularized filamentous cells.

6. Once the inter-cellular regions of the filamentous scaffold are determined to be adequately populated with the non-filamentous organism, creating a composite cellular mass, the composite cellular mass is extracted from the bioreactor vessel and passaged to post-processing.

Static Solid State-Submerged (SSSS) Cultivation for Production of Composite Cellular Masses

1. Solid substrate is prepared with appropriate asepsis and supplemental nutrition to support metabolism and growth of a given filamentous organism, filamentous organism inoculum introduced to the prepared substrate creating an inoculated substrate, and the inoculated substrate loaded into the bioreactor vessel. An example substrate for Laetiporus spp. would be hardwood chips supplemented with 20% wheat bran, which is pressure sterilized at 15 psi for 1 hour.

2. The inoculated substrate is incubated under conditions specifically selected to affect expression of a specific three-dimensional filamentous network morphology, which occurs external to the solid substrate mass creating a cohesive filamentous network which may be isolated from the solid substrate mass. Such incubation conditions are described in U.S. Ser. No. 16/190,585.

3. Example 001 steps 3-6.

Stirred Submerged-Submerged Cultivation for Production of Composite Cellular Masses

1. Filamentous organism inoculum is introduced into a bioreactor vessel containing a liquid medium prepared with appropriate asepsis and nutrition (as per Example 1) for cultivation of the given filamentous organism, creating a first inoculated media. The rate of addition of the filamentous organism inoculum is adjusted to target specific resultant filamentous pellet sizes optimized for downstream texture and cell adhesion to support growth, and media preparation and inoculation are performed to target an optimal media viscosity of 150 centipoises for maintenance of dissolved oxygen for filamentous organism cultivation.

A generic example of the rate of addition would be an 8% inoculation rate (vol/vol cell suspension inoculum to liquid medium) with the cell suspension prepared to at least 75% turbidity at OD 590 nm. The inoculum rate that was reduced to practice was an aliquot of 5×104 cells that were resuspended in 25 μL of fresh culture medium and were seeded onto scaffolds that had been immersed in medium and then compressed to expel the liquid.

2. Stirred incubation of the inoculated media is performed with conditions and stir rates selected to affect expression of a specific three-dimensional filamentous pellet morphology. The stirring is such as to maintain pellets opposed to breaking matts into pellets. The inoculum are individual fragments that further pelletize under stirred incubation conditions.

3. Example 001 steps 3-6

Stirred Submerged-Drip Cultivation for Production of Shaped Filamentous Structures

1. Example 003 steps 1-2

2. Application of inoculated media to surface of preformed shape representative of final desired product by sterile drip-application over the course of a number of days until a well formed mycelial sheet is grown on the surface of the shape

3. Extraction of mycelial sheet from shape surface with retention of shape as either a 2-D shell or a thicker 3-D tissue mat.

Submerged Co-Cultivation of Filamentous and Non-Filamentous Organisms for Production of Composite Cellular Masses

1. Examples 001 and 003 in which a media is prepared that is appropriate for cultivation of both the filamentous and non-filamentous organisms, and inoculum of each organism is introduced to the media simultaneously. Such a media could include potato dextrose broth, which supports both a filamentous fungus and a single celled bacterium.

2. Examples 001 and 003, in which incubation is performed with conditions appropriate for the cultivation of both filamentous and non-filamentous organisms, for example, at temperatures between 27° C. and 37° C., the upper threshold being appropriate for mammalian tissue culture and bacteria.

SSSS Cultivation of Cellular Structure with Controlled Morphology

Method [002] is followed.

After step 2. The following steps occur:
1. Moisture, signaling compounds such as hormones, minerals, and other molecules are directly deposited with micrometer precision on a grid (x,y) over the surface of the growth medium. While this embodiment contemplates the use of a printhead (much like a 3D printer), deposition method may be via spray, air conveyance, or any other method which allows precise deposition of material across the surface of a planar growth part. Molecules that both enhance growth, modify growth, and retard growth are contemplated. The addition of other living cells at this stage is contemplated and may either provide further in-situ molecule or signaling synthesis (e.g. time-delayed molecule synthesis post deposition) and/or become embedded into the growth of the part.
2. The rate of the deposition can be calibrated to match the growth rate of the organism in the y direction. Ideally, the entire surface of the part can be treated prior to additional upward tissue expansion (e.g. entire surface treatment can occur prior to a cell division of one hyphal length). The rate of deposition can also be arbitrarily slow so as to only allow one pass during an entire growth cycle. Deposition rate is selected based on the ultimate feature resolution desired and will often sit between these two extremes.
3. During each cycle, n, of hyphal growth in the z axis (measured in microns or hyphal lengths), the print head passes over the surface of the part in an x,y grid. Each x,y cell receives a precise dose of liquid which influences the tissue morphology & metabolism. Influenced tissue morphology and metabolism includes, but is not limited to, hyphal branching rate, cell wall thickness, types of hyphal tissue created, types of proteins and compounds excreted during hyphal growth, and direction of hyphal extension. Grid spacing can be selected at a minimum to match one hyphal unit (e.g. microns by microns cell size) or upwards to relatively large divisions (e.g. 1 mm×1 mm resolution). Resolution is selected based on the precision required for the grown tissue. Fine features, such as as scaffolding for capillaries, may require a very high level of resolution, where-in bulk features (creating a zone of higher density tissue in a structural element) may require relatively low resolution of deposition control.
4. Step 3 is repeated until the entire desired pattern (x,y,z envelope) is imprinted upon the grown tissue or until the tissue reaches its maximum hyphal extension limit.
5. The use of an x,y axis is used to describe the printing process and this embodiment contemplates the print head would move in linear fashion from an origin of (0,0) to (x,y)
6. The tissue is extracted from the reactor and can be further post processed or used as is. Potential applications include patterning of hyphal tissue to match existing organ types for cellular scaffolding (e.g. lung, liver, kidney, and the like), and patterning of hyphal tissue to create pre-determined macro-geometric structures (e.g, a honeycomb pattern with areas of low density of mycelium as per FIG. 1 and thicker hexagonal walls of higher density of mycelium as per FIG. 3). The principle of this approach is to selectively control regions of mycelium growth to present varying densities across a surface in a predicted manner.

Organisms

1. Examples 001-005, in which the filamentous organism is a saprobic fungus of the phylum Basidiomycota, Ascomycota, Zygomycota, Chytridiomycota, or Glomeromycota.

2. Example 001-005, in which the filamentous organism is a fungus that produces a monomitic, dimitic, or trimitic mycelium. Also, dimorphic organisms that initially present as an individual yeast cell and are then induced to go filamentous may be used, an example of which is Aureobasidium pullulans.

3. Example 001-005, in which the fungus is one of an edible species and is generally considered safe for human consumption.

4. Examples 001-005, in which the filamentous organism is a fungus which produces one or more cellular structures such as generative hyphae, binding hyphae, coralloid binding hyphae, skeletal hyphae, pseudoparenchyma, pseudocarp, intercalary blastogenic cells, acropetal blastogenic cells, cell swelling, terminal conidiation, intercallary conidiation, oidiation, arthrosporulation, stroma, perithecia, conidiogenic cells, conidiophores, rhizoids, or rhizomorphs.

5. Examples 001-005, in which the filamentous organism is a fungus of genus Pleurotus, Ganoderma, Polyporus, Grifola, Lentinus, Lentinula, Trametes, Herecium, Agrocybe, Armillaria, Agaricus, Stropharia, Schizophyllum, Laetiporus, Lepista, Hypomyces, Inonotus, Pycnoporus, Fomes, Fomitopsis, Daedaleopsis, Piptoporus, Ischnoderma, Phellinus, Phaeolus, Sparassis, Tyromyces, Laricifomes, Panellus, Rhizopus, Phlebia, Phanerochaete, Dichomitus, Ceriporiopsis, Lepiota, Stereum, Trichoderma, Xylaria, Cordyceps, Hymenochaete, Hypsizygus, Flammulina, Coprinopsis, Coprinus, Morchella, Clitocybe, Cerioporus, Volvariella, Tremella, Calvatia, or Fistulina.

6. Examples 001-005, in which the non-filamentous organism is a cell of a chordate organism and may be mammal, fish, bird, reptile, or amphibian.

7. Examples 001-005, in which the non-filamentous organism is a plant cell.

8. Examples 001-005, in which the non-filamentous organism is a non-chordate and may be a mollusk or arthropod cell.

9. Examples 001-005, in which the non-filamentous organism is a myocyte, neuron, neuroglial cell, lung cell, fibroblast, chondrocyte, endothelial cell, osteocyte, osteoblast, adipocyte, or stem cell.

10. Examples 001-005, in which the non-filamentous organism is a bacterium, yeast, algae, filamentous fungus, nucleic acid based lifeforms (virus, bacteriophage) or a mycoplasma.

11. Examples 001-005, in which the non-filamentous organism is a cell of a coral or shell structure.

Cultivation Paradigm Variations
Any of the below can be employed with Examples 001-005:

1. Examples 001, 002, and 003 where incubation of both the first and second inoculated media occurs in a single batch in which all media components are expended within the incubation phase without further adjustment.

2. Examples of 001 and 003 (both first and second inoculated media) and 002 (second inoculated media), where incubation is performed using a fed-batch paradigm, in which nutrients (carbon, nitrogen, minerals, and pH adjustment) are periodically fed into the inoculated media, with spent media proportionally removed, based on active or periodic monitoring of set threshold conditions for the given nutrient concentrations.

3. Examples of 001 and 003 (both first and second inoculated media) and 002 (second inoculated media), where incubation is performed using a continuous feed paradigm, in which nutrients (carbon, nitrogen, minerals, and pH) are continuously adjusted based on a continuous monitoring of set conditions for the given nutrient concentrations.

4. Example 002, where solid-state cultivation of the filamentous organism occurs in a tray vessel which is incubated in a secondary vessel which provides controlled gas exchange and content, relative humidity, and temperature. In this paradigm, the three-dimensional extra-particle filamentous matrix extends from the top surface of the solid substrate from the tray.

5. Example 002, where solid-state cultivation of the filamentous organism occurs in an actively aerated packed-bed bioreactor vessel in which input air is conditioned to specific CO2, humidity, and temperature and passes through the solid-substrate matrix.

In this paradigm, a void space remains in the vessel within which the three-dimensional extra-particle filamentous matrix develops.

6. Example 002, where the three-dimensional filamentous matrix is isolated from the solid substrate matrix prior to decellularization.

7. Example 002, where the three-dimensional filamentous matrix is not isolated from the solid substrate matrix prior to decellularization, and the composite cellular mass is isolated from the solid substrate matrix at the conclusion of cultivation.

8. Examples 001, 003, and 005 in which the filamentous and non-filamentous organisms are cultivated in separate vessels (A and B, respectively) in parallel, and in which the non-filamentous cells are passaged from vessel A to vessel B, filtered through the filamentous organism network of vessel B, depositing non-filamentous cells throughout the filamentous cell network. Non-filamentous cells which passage completely through vessel B are reclaimed and passaged back to vessel A or vessel B. Flow of non-filamentous cells from vessel A to vessel B may be periodic or continuous, and may occur during or after filamentous organism network development in vessel B.

9. Examples 001-005, in which the filamentous organism scaffold is fully or selectively filled with a secondary biocompatible material such as agarose or gelatin gels. These gels do not provide inherent vasculature or structure, but do provide another lever of control for surface area and porosity, serve as a secondary cross-linking agent, assist in modulating the modulus of elasticity selectively within the filamentous scaffold, aid in initiating/directing cellular differentiation of adhered cell, as well as potentially bolster water uptake and retention.

10. Examples 001-005, in which the filamentous organism scaffold is fully or selectively imbibed with growth factors for the non-filamentous organism. The growth factors may be perfuse within the filamentous scaffold (naturally diffusing), encapsulated within a time-release device, or through the use of synthetic biology to express said compounds constitutively or through inducible DNA controlling sequences and mechanisms (i.e, temporal, thermal, availability feedback loops, etc.).

11. Example 001, in which the filamentous organism scaffold develops attached to, or is otherwise attached to, a solid support connected to a mechanical actuation device by one or more faces of the three-dimensional filamentous scaffold. During Example 001, steps 4-6, the filamentous organism scaffold is mechanically actuated during non-filamentous organism propagation within the filamentous scaffold, stimulating differentiation and propagation.

Modulation of Cultivation Conditions to Affect Different Three-Dimensional Fungal Scaffold Morphologies

1. Examples 001-005 in which the filamentous organism is a saprobic fungus, for example a Laetiporus species. The Laetiporus species is selected and cultivated under conditions favorable to producing a vegetative mycelium comprised of an isotropic matrix of discrete hyphae (Figure 001). For Laetiporus spp. an example would be incubation at 27° C. for 15 days via the media and paradigms described in examples 001-005.

2. The isotropic matrix of 1 may be modified to express galvanotropism and hyphal agglomeration increasing the average strand thickness with (Figure 002), or without (Figure 003), increased network fractional anisotropy, as well as express an ellipsoidal morphology (Figure 004) by any combination of increasing incubation temperature, e.g. to 37° C., increasing CO₂ content, e.g. to greater than 2%, addition of volatile organic compounds or paramorphogens, e.g. terpene, decreasing gas exchange rate, increasing supplementation of starch or other simple carbohydrates, increasing supplementation of fatty acids, adjusting the nitrogen supplement, for example, by supplementing with peptone, or supplementation with surfactants (such as Tween 80).

3. The isotropic matrix of 1 may have network crosslinking (the combined effect of branching, anastomosis, and hyphal entanglement) and/or cell volume density decreased by any combination of increasing incubation temperature, increasing CO₂ content, addition of volatile organic compounds or paramorphogens, decreasing gas exchange rate, decreasing starch or other simple carbohydrates, fatty acids or nitrogen supplementation, modifying supplementation of calcium, or supplementation with surfactants.

4. The isotropic matrix of 1 may have network crosslinking and/or cell volume density increased by any combination of decreasing incubation temperature; decreasing CO₂ content, for example, decreasing to 17°-22° C.; increasing gas exchange rate, for example, increasing the gas exchange rate such that CO₂ is maintained at atmospheric levels; increasing starch or other simple carbohydrate supplementation; supplementing with recalcitrant carbohydrates, such as cellulose; and modifying supplementation of calcium.

Propagation of Myocytes on a Filamentous Fungal Scaffold as an Alternative Meat

1. Per Examples 001-005 and 008-009 in which the filamentous organism is an edible fungal species per Example 007, such as a Laetiporus species.

2. Per Examples 001-005 and 008-009 in which the non-filamentous organism is a chordate myocyte of a bovine, avian (such as chicken), or fish cell line.

Production of Ground Meat Product Modifying Texture by Adjustment of Filamentous Scaffold Pellet Size

1. The process of Example 003 in which the filamentous organism is an edible fungal species (such as a Laetiporus spp.) which produces a floccose pellet morphology, and the non-filamentous organism is a cow (beef) myocyte.

2. Example 003 in which the inoculation rate of the Laetiporus species into the media is adjusted to target a specific textural quality of the resultant composite tissue mass. For instance, to target a coarse texture the inoculation rate would be decreased resulting in a larger pellet size, and ultimately a larger beef myocyte pellet. For example, the addition rate of Example 3 (8% v/v) may be reduced to 2%, or alternatively the 75% turbidity inoculum may be diluted.

Alternatively, to create a fine texture, the inoculation rate would be increased resulting in a smaller pellet size, and ultimately a smaller beef myocyte pellet. For example, the addition rate of Example 3 (8% v/v) may be increased to 16%, or alternatively the 75% turbidity inoculum may be concentrated to a higher cell density.

3. The resultant Laetiporus-beef myocyte composite tissue mass is applied as a ground beef replacement with “grind”, or texture, dictated by the tissue pellet size per 1 and 2.

4. steps 1-3 with alternative myocyte lines as per Example 007, steps 5-7.

Example 011 with Additional Filamentous Organism Regrowth into Specific Geometries

1. Example 011, except the fungal scaffold is not decellularized prior to cultivation of the beef myocyte, thus maintaining the viability of the fungal scaffold fraction.

2. After extraction of the fungus-beef myocyte composite mass, the mass is cast into molds of a defined geometry, for example a patty.

3. The molded fungus-beef myocyte composite mass is incubated under conditions appropriate for continued growth of the fungal fraction, leading to the discrete pellets binding together through filamentous extension into a cohesive mass of the given geometry. The final fungus-beef myocyte form is employed as a food product.

Production of Alternative Protein Matrix

1. Example 002, in which the filamentous organism is an edible species, such as Laetiporus, with the hyphal scaffold being aseptically extracted from the reactor or solid state substrate after step 2 and used, with or without further modification, as a food product.

Modifications of Alternative Protein Matrix
1. Example 010-013, where the harvested tissue is forced to express excess exocellular-mucilage through immersion in water, alteration of media nutrition, for example, supplementation with simple sugars and/or environmental conditions, for example, increasing the temperature to 37° C.
2. Example 010-013, where autolysis is induced in the living scaffold, to yield a more tender texture. For example, temperature induced autolysis maybe induced by increasing the incubation temperature to 40° C. for a short period at the conclusion of the incubation cycle (2-48 hours).
3. Example 010-013, where additional enzymes (e.g, chitinase, transglutaminases, proteases, glucanases, or the like) are applied to the extracted scaffold, or expressed (via synthetic biology) to modify the texture of the structure.
4. Example 010-013, where a secondary organism producing enzymes of interest is co-cultured upon the scaffold to produce in-vivo modification of the scaffold texture and structure. For instance, a mycoparasite, such as Trichoderma spp. or Mucor spp., which produce proteases, glucinases, and chitinases may be used as the secondary organism.
5. Example 010-013, where the harvested tissue is subjected to a corrosive compound (e.g., 1M HCl, 0.8M acetic acid [white vinegar], or the like), with or without heat, to alter the texture or porosity of the resultant structure.
6. Example 010-013, where the harvested tissue is subjected to a strong base, with or without heat, to remove acetyl groups from chitin, and/or alter the texture or porosity of the scaffold. An example of a method for chitin extraction is described at http://www.iglobaljournal.com/wp-content/uploads/2015/07/6.-Krishnaveni-Ragunathan-IGJPS-2015.pdf.
7. Example 010-013, where the harvested tissue is subjected to a known solvent for chitin (e.g., CaCl2 saturated methanol, ionic liquids, or the like), to alter texture or modify porosity
8. Example 010-013, where the harvested tissue is subjected to mechanical degradation to alter the natural texture, porosity, or density of the tissue (e.g., perforation, cutting, rolling, pressing, or the like)
9. Example 010-013, where the natural flavor compounds in the edible species are overexpressed through nutrition, synthetic biology, or environmental conditions (e.g., benzaldehyde in Pleurotus, phenylacetaldehyde in Suillus, anisaldehyde in Trametes, or the like)
10. Example 010-013, where another organism is cultivated upon the resulting matrix, where said organism produces desired flavoring compounds. (e.g., diacetyl [buttery] from lactic acid bacteria, pyrazine [roasted] and glutamates [meaty] from Corynebacterium glutamicum, or the like)
11. Example 010-013, where commercially available flavorings, fats, colors, heme, thickeners, sweeteners, acids, or the like are infused into the tissue scaffold to create a food product.
12. Example 010-013, where compounds are added to tissue or media during growth, to alter the end product's flavor, texture, or color (e.g., addition of glutamate in media, atomization of colorant with misters, atomization of natural flavor extracts, addition of forskolin to media to induce hyphal branching and alter finished texture, and the like)
13. Example 010-013, where the resulting tissue is fortified with vitamins and minerals to boost nutritional value, and/or replicate that of meat.
14. Example 010-013, where the growth media is amended, to vary the final nutritional profile of the tissue (e.g., addition of amino acids to increase fatty acid concentration, mineral atomization or addition, vitamin supplementation, proteins, and the like)
15. Example 010-013, in which the hyphal scaffold is imparted with a defined grain by selection of fungal species or cultivation conditions that result in galvanotropism and hyphal agglomeration per Examples 007 & 009.
16. Example 010-013, in which the hyphal scaffold is imparted with a more delicate and fracturable texture by selection of fungal species or cultivation conditions that result in intercallary and/or terminal conidiation per Examples 007 & 009.
17. Example 010-013, in which the hyphal scaffold is imparted with a more delicate and fracturable texture by selection of fungal species or cultivation conditions that result in conidiation per Examples 007 & 009.
18. Example 010-013, in which the hyphal scaffold is imparted with a more delicate texture by selection of fungal species with a monomitic, dimitic, or otherwise a hyphal morphology free of structural or skeletal hyphae per Examples 007 & 009.
19. Example 010-013, in which the hyphal scaffold is imparted with a uniform texture by selection of fungal species or cultivation conditions that result in an isotropic hyphal morphology per Examples 007 & 009.
20. Example 010-013, in which the hyphal scaffold is imparted with a tough or chewy texture by selection of fungal species with a trimitic, dimitic, or otherwise a hyphal morphology that includes structural or skeletal hyphae per Examples 007 & 009.
21. Example 010-013, in which the hyphal scaffold is imparted with a reduced cohesiveness and/or cohesiveness of mass by selection of fungal species or cultivation conditions that result in blastogenesis or pseudoparenchyma per Example 007 & 009.
22. Example 010-013, in which the hyphal scaffold is imparted with a greater density by modification of cultivation conditions to increase hyphal branching, anastomosis, and/or entanglement per Examples 007 & 009. Alternatively, the hyphal scaffold may be imparted with a reduced density by modification of cultivation conditions to decrease hyphal branching, anastomosis, and/or entanglement per Examples 007 & 009.

Embodiment: Bovine Meat

As per methods 9 and 10, in which the filamentous scaffold is a saprophytic fungus of the genus Laetiporus grown in conditions described therein, where the secondary non-filamentous organism is comprised of myoblasts of the genus Bos, creating a three-dimensional edible fungal scaffold, imbibed with propagated bovine meat cells, to be used as a food product.

Embodiment: Seafood

As per method 005, in which the filamentous scaffold is a saprophytic fungus of the genus Rhizopus grown in conditions described therein, where the non-filamentous organism is a myoblast of the phylum Mollusca, creating a three-dimensional edible fungal scaffold, imbibed with propagated mollusk meat cells, to be used as a food product or structural material.

Embodiment: Neutral Alternative Protein

As per method 013, in which a solid billet of vegetative hyphae of the genus Herecium is extracted without any inoculation with non-filamentous organisms. This scaffold is post-processed per 014, with an application of chitinase from papaya extract to improve texture, then heated in 1 molar acetic acid to further modify texture. The resultant tissue is then imbued with vegetable fat, marinated in autolyzed yeast, smoke flavor, tomato extract, spices, and fortified with minerals and vitamins. Then, the tissue is cooked until crispy, to produce a non-animal bacon-like product.

Embodiment: Flavored Alternative Protein

As per method 002 in which a solid billet of vegetative hyphae of the genus Flammulina is grown with added glutamate in media to impart umami and essential dietary minerals and to fortify the resulting tissue. After initial growth, the filamentous scaffold is then inoculated with lactic acid bacteria or yeast to produce diacetyl in situ, lending a butter-like flavor and aroma. The tissue is then harvested, imbued with vegetable fats and proteins, and cooked. Resulting in a food item, with natural flavoring and meat-like texture and properties.

Embodiment: Lung

As per method 006 in which the filamentous scaffold is a saprophytic fungus of the genus Ganoderma grown in conditions described therein and the secondary non-filamentous organism is bronchiolar epithelium cells. The filamentous scaffold is grown under conditions described in 009, in which agglomerative galvanotropic growth is elected, to mimic the vascular nature of alveoli, allowing the secondary cells to form a structured three-dimensional mass of tissue.

Embodiment: Brain, Using Rhizomorph to Support Axon Growth

As per methods 002 and 006 in which the filamentous scaffold is a saprophytic fungus of the genus Armillaria grown in conditions described therein, selecting growth parameters that express rhizomorphic growth, a highly anisotropic, galvanotropic, cord-like morphology. These cord-like structures are then inoculated with a secondary non-filamentous organism such as mammalian neural stem cells, to support axon-like cell growth, along a naturally-structured scaffold.

Embodiment: Beauty Applicator

As per method 003, in which a solid billet of vegetative hyphae of the genus Laetiporus is incubated under day/night light cycles and increased air exchange, which elicit the expression of exogenous pigmentation of the hyphal scaffold. This scaffold is then post processed as per 014, with the impregnation of beneficial fatty acids, such as lauric acid, to improve application smoothness and foam rigidity, resulting in a makeup applicator like foam with naturally produced pigments that can be applied to the skin.

Embodiment: Disposable Paint Brushes

As per method 013, in which a solid billet of vegetative hyphae of the genus Ganoderma is extracted without any inoculation with non-filamentous organisms and post processed as per 014 with a 10% hydrogen peroxide soak to exfoliate the tissue and increase porosity/absorptive capacity, resulting in a biodegradable foam billet that can be used to replace traditional polymeric foam brushes.

Embodiment: Sensing

As per method 002, in which the filamentous scaffold is a saprophytic fungus of the genus Rhizopus grown in conditions described therein, where the secondary non-filamentous organism is comprised of electroactive bacteria, such as the genus Shewanella, and wired to a current collector and a voltmeter, for monitoring of water contamination of sewage, runoff, and/or pollutants.

Embodiment: Waste Water Treatment

As per method 002, in which the filamentous scaffold is a saprophytic fungus of the genus Ganoderma that is grown in conditions described therein, where the secondary non-filamentous organism is comprised of a hybrid culture of Cyanobacteria, for oxygen production, and Betaproteobacteria for organic treatment, resulting in a biodegradable cassette that can be used and/or produced in-field for treatment of latrines, disaster relief, or the like.

Embodiment: Antibiotic Sponge

As per method 002, in which the scaffold is comprised of a saprophytic fungus of the genus Trametes (with or without drug resistance) that is grown in the conditions described therein (with or without antibiotics), where the panel is either then sterilized, and imbibed with antibiotics, or inoculated and incubated with an antibiotic producing organism, then sterilized and packaged. This biodegradable 3-D scaffold can then be adjusted to size and used for implantation, for internal antibiotic treatment of cavity wounds, or use as a biodegradable temporary wound dressing for trauma or disaster relief.

Embodiment: Absorption/Dispersal
1. Method 002 is followed, followed by imbibement of desired antibiotic for medical treatment
2. Tissue is then rendered flat by cold compression to form an essentially 2-D shape
3. Flattened tissue is desiccated to preserve tissue quality
4. Imbued 2-D tissue is used in small space insertion.
5. Expansion within the space beyond the small insertion space is affected with specific design as imparted by lattice memory where 2-D flat sheets can fit through small holes/incisions, rehydrate, and expand to original morphology to fill the largely inaccessible space
6. Expanded tissue fulfills role as interior diffusive scaffold for antibiotics for internal surgery

Embodiment: Biodegradable Wound Dressing for Damaged Tree Limbs

1. Method 002 is followed

2. Resultant tissue is rendered vitally inert through heat application

3. Tissue is imbued with antifungal and antibiotic treatments specific to injured tree specie

4. Tissue is applied to wound surface for an indeterminate amount of time, until the tissue mat is degraded or overgrown

Embodiment: An Implantable Fungal Scaffold with Semiconducting Properties

1. Example 001 steps 1-2, Example 002 steps 1-2, or Example 003 steps 1-2, in which the filamentous organism is Schizophyllum commune or Morchella spp, and is a strain of which produces indigotin.

2. MgSO4, 7H2O is supplemented at a rate of 0.1-1% (mass/volume) into the culture media of Examples 001-003.

3. Incubation occurs under environmental conditions appropriate for supporting metabolism and growth of the selected fungal strain, during which biosynthesis of exogenous indigotin occurs, resulting in indigotin deposition on the exterior of the fungal hyphae. In this case, the extent of indigotin biosynthesis and exogenous deposition may be modified by the MgSO4, 7H2O supplementation rate per step 2.

4. The resultant three-dimensional hyphal scaffold, with exogenous indigotin or melanin coating of the hyphal cells, is isolated for downstream use as an implantable, biocompatible, semi-conducting material.

5. The semi-conducting hyphal scaffold of step 4 is passaged to Examples 001-005 steps 3 forward.

Embodiment: Example 028 in which Exogenous Indigotin is Deposited onto a Secondary Surface

1. Example 028 steps 1-3 in which an additional cell-type or material co-occupies the culture medium with an indigotin producing fungal strain.

2. Step 1, in which the additional cell-type is the non-filamentous species of Examples 001-005.

3. Step 1, in which the additional material co-occupying the culture medium is an organic substrate.

4. Step 1, in which the additional material co-occupying the culture medium is an inorganic substrate.

Embodiment: Implementation of Static Submerged Filamentous Fungus Scaffolding Reactor Unit in a Perfusion Reactor System to Produce an Alternative Meat Product

Method [001] is followed.

During step 1, the fungus selected is one of an edible species, for example Laetiporus spp., and specifically, Laetiporus sulphureus, which is inoculated into a vessel containing a culture medium comprised of corn steep solids, glucose, potassium phosphate, magnesium sulfate, and pH adjusted to between 5.5-6.5. The vessel is designed such as to allow flow of media through the vessel, and is implemented as a scaffold tray unit within a perfusion bioreactor system in which a suspension bioreactor for beef myocytes feeds directly to the scaffold tray unit in which the filamentous fungus is to be cultivated. The vessel further contains a sparger and diffuser in the center of the scaffold tray vessel volume, running the length of the scaffold tray vessel.

During step 2, incubation of the Laetiporus spp. inoculated media occurs without flow from the beef myocyte suspension reactor unit under static conditions with dissolved oxygen levels maintained by an filtered air feed through the sparger and diffuser, allowing for a contiguous hyphal network to develop within the scaffold tray vessel, which further grows into the sparger and diffuser, anchoring the contiguous hyphal network in place. Scaffold tray bioreactor operation may be performed as a batch, fed-batch, or continuous-feed process. During this stage the dissolved oxygen levels, light exposure, temperature, and media components may be modified according to Method [009].

Step 3 is followed.

During step 4, the decellularization chemistry is replaced with fetal bovine serum containing growth factors for the beef myocytes, and flow of beef myocytes from the suspension bioreactor unit to the filamentous fungus scaffold tray reactor unit is initiated. The media may be further supplemented with polylactic acid, polycaprolactone, or polyglycolic acid to assist with adhesion of beef myocytes to the decellularized filamentous fungal cells (hyphae).

Referring to Figure 005, during steps 5 and 6, incubation occurs with either continuous or periodic flow of fetal bovine serum and suspended beef myocytes (6, 7) between the filamentous scaffold tray unit (3) and the myocyte suspension reactor unit (1), during which myocytes (2) attach to the hyphal scaffold (5) within the scaffold tray reactor unit (3) anchored to the sparger and diffuser (4) and replicate, resulting in agglomerations of myocytes within the inter-hyphal volume of the filamentous network, creating a composite cellular mass of hyphae and myocytes. This composite cellular mass is then extracted for post-processing as an alternative meat.

Embodiment: Implementation of a Static Solid State-Submerged Filamentous Fungus Scaffolding Reactor Unit in a Perfusion Reactor System to Produce an Alternative Meat Product

Method [002] is followed.

During step 1, a solid substrate is prepared with corn stover, starch, cereal grains, and is inoculated with an edible fungal species such as Laetiporus spp., and specifically, Laetiporus sulphureus, The prepared substrate is filled into a Type I tray bioreactor system, such as described in Mitchell et al. (Eds) Solid-State Fermentation Bioreactors, Springer-Verlag Berlin Heidelberg (2010), and loaded into an incubation vessel with temperature, light, carbon dioxide, oxygen, relative humidity, and vapor deposition control.

During step 2, incubation conditions are maintained at 5% carbon dioxide and near 100% relative humidity. Additionally, Method [006] may be followed during this stage to effect specific heterogeneous morphologies. A negatively gravitropic extra-particle fungal hyphal matrix develops from the inoculated substrate, which is further modified during growth via modulation of light, oxygen, carbon dioxide, relative humidity, or vapor deposition rate per Method [009]. The extra-particle hyphal matrix develops into a contiguous mass, which is isolated from the solid substrate for post-processing.

Method [001] step 3 is followed.

The decellularized hyphal scaffold is transferred to a scaffold tray vessel within a perfusion bioreactor system. Steps 4-6 of Embodiment 030 are followed.

Embodiment: Implementation of Submerged Co-Cultivation of a Filamentous Fungal Matrix and Beef Myocytes in a Perfusion Bioreactor System for Production of an Alternative Meat Product

Method [001] is performed according to the modifications of Method [004].

During Method [001] step 1, a culture medium is prepared and inoculated within a tray vessel reactor implemented in a perfusion bioreactor per Embodiment 030.

During Method [001] step 2, incubation of the Laetiporus spp within the scaffold tray vessel occurs according to Embodiment 030 until filamentous growth of Laetiporus spp has been established and has become anchored in the sparger and diffuser.

According to Method [005] step 1, flow from the beef myocyte suspension reactor per Embodiment 030 is initiated through the developing fungal scaffold within the scaffold tray vessel. At this point, the media is comprised of nutrients supportive of both propagation of Laetiporus spp and the beef myocytes, and may include corn steep solids, glucose, potassium phosphate, magnesium sulphate, fetal bovine serum, beef myocyte growth factors, polylactic acid, polycaprolactone, or polyglycolic acid, and pH adjusted to between 5-7. According to Method [004] step 2 both P. ostreatus and beef myocytes develop in parallel, producing a composite cellular mass according to Embodiment 030 steps 5 and 6.

Embodiment: Use of Imbued Tissue to Produce Crystal Structure Deposition
1. Method [002] is followed to produce a mycelial tissue sheet.
2. Tissue sheet is compressed to flatten and evacuate residual moisture from intercellular pores
3. Tissue is then subjected to heavily mineralized liquid and allowed to absorb said liquid to full saturation
4. Tissue is deposited in location specified as mineral deposition zone
5. Tissue desiccates ambiently and acts as a time release of mineralized residue previously held within intercellular pore spaces

Embodiment: Production of Vasculature

Method [001] is performed according to Embodiment 030, where the filamentous organism is a rhizomorphic strain of Armillaria gallica, and the non-filamentous organism is comprised of any combination of endothelial cells, myocytes, and fibroblasts.

During steps 1 and b 2 A. gallica fills the volume of the scaffold tray bioreactor unit with a matrix of rhizomorphs ranging from <1 mm to 5 mm in diameter.

During steps 4-6 endothelial cells, myocytes, and/or fibroblasts attach to and propagate along the surface of the rhizomorphs, forming a cohesive outer cellular layer or sleeve.

During post-processing, a sleeve of endothelial cells, myocytes, and/or fibroblasts are isolated from the underlying A. gallica rhizomorph by any combination of chemical lysis or mechanical separation.

Embodiment: Grown Tools

Examples 001-005, in which the filamentous scaffold is grown into predetermined shapes, such as small hand tools (hammer). The scaffold is co-cultured with non-filamentous cells (i.e., yeast, bacteria, and the like), which adhere and deposit polymers, metals, keratin, calcite, or spider silk onto the scaffold matrix, thus providing enhanced mechanical strength, and structural stability. The synthesis and deposition of compounds can the enhanced through strain engineering.

Embodiment: Alternative to Mammalian Meat (Yeast)

Examples 001-005, in which the filamentous scaffold is co-cultured with yeast cells which are allowed to adhere to either decellularized or intact cellular scaffolds. Yeast will be cultivated in co-culture or independently (fermenter B, FIG. 5), and used as an alternative to mammalian cells to circumvent cellular cultivation with expensive bovine serums, and surface attachment requirements.
1. Yeast or the filamentous organism can be genetically engineered to enhance binding affinity to the scaffolds (i.e., chitin, hydrophobin binding motifs).
2. Yeast can be engineered to express meat based flavors and properties (i.e. heme, fats, pigments). The expression of these compounds can be constitutive throughout cultivation/assembly, or induced at desired times during the cellular assembly process for optimized expression and impact.

Embodiment: Engineered Fungal Edible Meats

Examples 001-005, in which the filamentous scaffold organism is genetically engineered to possess desired characteristics of natural meat flavor, color, texture, and smells (i.e., heme, fats, pigments).

Examples of how the organism can be genetically engineered include methods of up-regulating existing genes to enhance the composition of glutamic acid within the fungal tissue to provide a more umami flavor profile, or to do the same for pigmentation pathways such as melanin induction. Further, the organism can be engineered to “knock-out” or eliminate specific genes that lead the differentiation of the mycelium into a mushroom thus amending or limiting texture changes. Finally, the organism can be engineered to introduce a promoter and gene cassette for a molecule from another organism, such as heme.

Embodiment: Therapeutic Delivery

Examples 001-005, in which the filamentous scaffold organism and/or co-cultured non-filamentous cells are used to deliver therapeutics to implanted tissues (i.e. dermal, subcutaneous, intramuscular, and the like). In this embodiment, the therapeutic is produced by the non-filamentous cells and encapsulated within the filamentous scaffold. The release of the therapeutic can be related to concentration differentials between the scaffold and the surrounding tissues (e.g., Fickian or Non-Fickian Diffusion). The therapeutic can also be released to surrounding tissues as the scaffold is degraded or incorporated into said tissues.

1. Filamentous organism can be genetically engineered to express or have cell surface binding/release affinity for the delivered therapeutic.

2. Non-filamentous organisms can be genetically engineered to express or have binding/release affinity for the delivered therapeutic.

3. Therapeutic can be released by constitutive compound synthesis, or a temporal base degradation release profile (therapeutic binding affinity)

4. Both (1-2) cells can be engineered to detect the titer of the therapeutic in the implanted tissue or extracellular matrix, thus regulating the synthesis or release of the therapeutic.

Embodiment: Self-Protective Scaffold (Sense-Response)

Examples 001-005, in which the filamentous scaffold organism and/or co-cultured non-filamentous cells are genetically programmed to sense microbial contaminants and pathogens (E. coli, Staph).

In this embodiment, non-filamentous strains (i.e., bacteria, yeast) are genetically engineered to contain multiple sensors integrated into the genome that respond to signals associated with microbial contaminants such as bacteria and fungi that represent human health threats, or are detrimental to the structural integrity of the filamentous scaffold matrix. Multiple sensors and specificity will be achieved through the integration of these sensors via genetic logic gates in order to positively identify the strain.

Engineered non-filamentous organisms would be co-cultured with the scaffold and maintained as living cells to provide an active immunity against infection. These co-cultured strains will respond to particular patterns of quorum molecules associated to the contaminants, along with other indicators, and use a classifier circuit to select the correct antibiotic/antifungal to produce.
1. Food safety—enable foodstuffs to identify the presence of pathogenic microbes (i.e., E. coli, salmonella, Clostridium, etc.), and initiate a response by expressing and secreting species specific antibiotics to suppress or kill said contaminants.
2. Implantation—enable the living cellular scaffold matrix to sense the presence of problematic microbial contaminants (i.e., Staphylococcus aureus, etc.) and initiate a response to suppress or kill invading microbes before and after surgical implantation.

Embodiment: Living Scaffold Utilities

Examples 001-005 and [007] Organisms, Enable filamentous and non-filamentous cells to express limiting nutrients need for successful cultivation and surgical implantation scaffold viability.
1. Position microbes in co-cultivation microbiome that are natural growth promoting organisms and target for enrichment.
2. Genetically engineer [007] microbes to promote enhanced system wide growth in the cultivation/scaffold assembly process,
3. Genetically engineer [007] microbes to support scaffold health and sustainability once implanted as a medical device.

Embodiment: Tissue Generated/Scaffold Removal

Examples 001-005, in which the filamentous scaffold organism is used to support the adhesion and differentiation of co-cultivated cells (i.e., myoblasts) to establish functional tissue forms i.e., medical devices, foodstuffs, and the like.
1. Scaffold remains part of the formed tissue throughout the intended usable life of the tissue (i.e., medical implantation, foodstuff), and continues to provide structural support or fosters viability and/or growth of attached cells.
2. Scaffold is “removed” from the final tissue form. The filamentous scaffold is degraded in vitro or in situ using enzymes, compounds, pH, thermochemical applications, and the like.
3. Scaffold degrading enzymes, compounds, small molecules, or other Substrates can be introduced during the formation of the final tissue (i.e., within the fermentor) by being fed into the reactor from an external source, or expressing said degrading agents from co-cultured cells (adhered or free floating).
4. Degrading agents could also be produced by microbes in a secondary reactor. Agents could be isolated and purified, or used within the cell suspension, then transferred to the final tissue to remove or degrade the filamentous scaffold matrix leaving behind “pure tissue” i.e., myoblasts, and the like.

The invention thus provides methods of generating mycelial scaffolds that leverage the phenotypic plasticity of filamentous fungi to produce fungal scaffold materials with specifically targeted network morphologies.

The invention also provides mycelium scaffolds for implementation in perfusion bioreactor systems for cell-based meat technologies.

The invention also provides mycelium scaffolds that provide an optimized fibrous, complex substrate for adhesion, propagation, and agglomeration of mammalian cells in suspended or submerged culture.

The invention also provides methods to produce biocompatible and biodegradable mycelium scaffolds with unique plasticity of manufacture, allowing for porosity and structure to be uniquely tunable for biomedical applications.

What is claimed is:

1. A method of generating a mycelial scaffold comprising the steps of
placing a substrate of a nutritive substrate and a fungus in a defined environment with a temperature of from 85° F. to 95° F. and a carbon dioxide content of from 3% to 7% of the environment; said fungus characterized in being a. biocompatible species;
incubating said substrate in said environment to induce mycological biopolymer growth from said substrate without producing a stipe, cap or spore therein; and
removing the growth of mycological biopolymer from said substrate as a one piece self-contained billet.

2. A method as set forth in claim 1 wherein said fungus is a filamentous organism and which further comprises the step of introducing a non-filamentous organism into said substrate for incubation and co-cultivation of said filamentous organism and said non-filamentous organism into said billet.

3. A method as set forth in claim 2 wherein said non-filamentous organism is a chordate myocyte of one of a bovine, avian and fish cell line.

4. A method as set forth in claim 2 wherein said filamentous organism is of the genus Laetiporus spp, and said non-filamentous organism is a chordate myocyte of a bovine.

5. A method as set forth in claim 2 wherein said filamentous organism is a saprophytic fungus of the genus Rhizopus. and said non-filamentous organism is a myoblast of the phylum Mollusca.

6. A method of generating a mycelial scaffold comprising the steps of
inoculating a filamentous organism into a liquid medium in a bioreactor vessel containing nutrition for cultivation and growth of said organism,
incubating said inoculated medium for a time sufficient for the growth of a filamentous network within said vessel,
thereafter decellularizing said filamentous network to form a decellularized filamentous scaffold within said vessel;
adding a second liquid medium to said vessel for cultivation of a selected cell line of a non-filamentous organism;
inoculating a non-filamentous organism into said second liquid medium, and
incubating said inoculated second medium for a time sufficient for the growth of said non-filamentous organism into said cellularized filamentous scaffold to from a composite cellular mass.

7. A method of generating a mycelial scaffold comprising the steps of
inoculating a filamentous organism into a liquid medium in a bioreactor vessel containing nutrition for cultivation and growth of said organism at a rate of inoculation to target specific resultant filamentous pellets sizes optimized for downstream texture and cell adhesion to support growth;
maintaining a viscosity of said inoculated liquid medium at a degree sufficient to maintain dissolved oxygen for filamentous organism cultivation into a filamentous network; and
stirring said inoculated medium at a degree sufficient to affect expression of a specific three-dimensional filamentous pellet morphology from said filamentous network.

8. A method as set forth in claim 7 further comprising the steps of applying said inoculated medium to a surface of a preformed element in a drip-wise manner for a time sufficient for a mycelia sheet to form on said surface. and removing said mycelial sheet from said element.

9. A method as set forth in claim 7 further comprising the step of introducing a non-filamentous organism into said liquid medium in the bioreactor vessel for incubation and co-cultivation of said filamentous organism and said non-filamentous organism.

10. A method as set forth in claim 9 wherein said filamentous organism is an edible fungus that produces a floccose pellet morphology and said non-filamentous organism is a cow myocyte.

11. A method as set forth in claim 10 wherein said filamentous organism is Laetiporus spp. and is inoculated into said liquid medium at a rate to target a specific textural quality of the resultant filamentous pellet morphology wherein decreasing said rate effects a larger pellet size and increasing said rate effects a smaller pellet size.

12. A method as set forth in claim 7 further comprising the steps of adding a second liquid medium inoculated with a non-filamentous cow myocyte to said vessel prior to said step of stirring for cultivation of said cow myocyte therein, and incubating said inoculated second medium for a time sufficient for the growth of said cow myocyte into said filamentous network prior to said step of stirring to form a composite cellular mass.

13. A method of generating a mycelial scaffold comprising the steps of
Inoculating a filamentous organism into a substrate;
loading the inoculated substrate into a bioreactor vessel containing nutrition for cultivation and growth of said organism;
incubating said inoculated substrate for a time and under conditions sufficient to affect expression of a specific three-dimensional filamentous network morphology external to said substrate; and
depositing at least one layer of a selected material onto said filamentous network morphology during expression of said filamentous network morphology to impart predetermined characteristics to said filamentous network morphology.

14. A method as set forth in claim 13 wherein said selected material is one of a hormone and a mineral.

15. A method as set forth in claim 13 which further comprises the steps of removing said filamentous network morphology from the bioreactor vessel and patterning said filamentous network morphology with a predetermined shape.

16. A method of generating a mycelial scaffold comprising the steps of
inoculating a filamentous organism into a growth medium in a bioreactor vessel containing nutrition for cultivation and growth of said organism,
incubating said inoculated medium for a time sufficient for the growth of a filamentous network within said vessel into a self-contained scaffold; and
introducing a secondary biocompatible material into said scaffold to impart a desired characteristic to said scaffold.

17. A method as set forth in claim 16 wherein said secondary biocompatible material is one of agarose and gelatin.to provide a secondary cross-linking agent.

18. A method as set forth in claim 16 further comprising the steps of
applying chitinase from papaya extract to said scaffold to improve texture,
thereafter heating said scaffold in 1 molar acetic acid to further modify texture,
thereafter imbuing said scaffold with vegetable fat,
marinating said scaffold in autolyzed yeast, smoke flavor, tomato extract and spices,
fortifying said marinated scaffold with minerals and vitamins, and
cooking said fortified scaffold until crispy to produce a non-animal bacon-like product.

19. An apparatus for generating an alternative meat product comprising
a scaffold tray unit for containing a culture medium inoculated with filamentous organism and for growth of a contiguous hyphal network therefrom;
a sparger in said scaffold tray unit for delivering air to said tray unit and for growth of the hyphal network thereinto;
a diffuser connected to said sparger in said scaffold tray unit for diffusing air onto said tray unit and for growth of the hyphal network thereinto; and
a myocyte suspension reactor unit for beef myocytes in communication with said scaffold tray unit to deliver a flow of beef mycocytes into the hyphal network in said scaffold tray unit.

20. A method for generating an alternative meat product comprising
growing a hyphal network from a culture medium inoculated with a filamentous organism within a scaffold tray unit;
delivering air to said tray unit for growth of the hyphal network therein;
thereafter decellularizing said hyphal network to form a decellularized filamentous scaffold within said scaffold tray unit;
introducing a flow of fetal bovine serum containing growth factors into said filamentous scaffold;
thereafter delivering a flow of beef myocytes into said decellularized filamentous scaffold for attachment to and in said filamentous scaffold to form a composite mass of hyphae and myocytes; and
processing said mass as an alternative meat product.
